(19) United States
(12) Patent Application Publication
(10) Pub. No.: US 2021/0276007 A1
(43) Pub. Date: Sep. 9, 2021

(54) SAMPLE COLLECTION DEVICE AND SAMPLE PREPARATION DEVICE

(71) Applicant: PHC HOLDINGS CORPORATION, Tokyo (JP)

(72) Inventor: Shingo OOTANI, Toon-shi, Ehime (JP)

(21) Appl. No.: 17/256,496

(22) PCT Filed: Jun. 27, 2019
(86) PCT No.: PCT/JP2019/025720

§ 371 Date: Dec. 28, 2020

(30) Foreign Application Priority Data
Jun. 29, 2018 (JP) 2018-124219

(51) Int. Cl. B01L 3/00; G01N 33/72; A61B 5/15

(52) U.S. Cl. CPC B01L 3/5023; G01N 33/723; G01N 33/726; B01L 2400/0406; B01L 2200/16; B01L 2300/041; A61B 5/150755

(57) ABSTRACT

A sample collection device includes a grip portion and a collection portion connected to the grip portion, wherein the collection portion includes a first surface and a capillary having a groove shape provided on the first surface and formed with openings at opposite ends thereof.

### TECHNICAL FIELD

The present application relates to a sample collection device and a sample preparation apparatus.

### BACKGROUND ART

A sample collection device for collecting a minute amount of a liquid is used for analysis of a particular component in the blood, or the like. Patent Document No. 1 shows an example of such a sample collection device. The sample collection device disclosed in Patent Document No. 1 collects a liquid by utilizing a capillary force.

### CITATION LIST

### Patent Literature

Patent Document No. 1: Japanese National Phase PCT Laid-open Publication No. 2007-527537

### SUMMARY OF INVENTION

### Technical Problem

With the conventional sample collection device, it has sometimes been difficult to discharge a liquid sample retained therein. The present disclosure provides a sample collection device with which it is easy to discharge a collected liquid sample.

### Solution to Problem

A sample collection device in one aspect of the present disclosure includes a grip portion and a collecting portion connected to the grip portion, wherein the collecting portion includes a first surface, a second surface and a third surface that are adjacent to the first surface with the first surface sandwiched therebetween, and a capillary that has openings on the first surface, the second surface and the third surface.

### Advantageous Effects of Invention

In one aspect of the present disclosure, there is provided a sample collection device with which it is easy to discharge a collected liquid sample.

### BRIEF DESCRIPTION OF DRAWINGS

FIG. 1 is a perspective view showing one embodiment of a sample collection device.

FIG. 2 is a front view showing the sample collection device as seen from the tip.

FIG. 3 is a side view showing a tip portion of the sample collection device on an enlarged scale.

FIG. 4 is a side view showing the tip portion of the sample collection device on an enlarged scale.

FIG. 5 is an exploded perspective view showing a sample preparation apparatus.

FIG. 6 is a cross-sectional view, taken parallel to the longitudinal direction, showing a reagent container of the sample preparation apparatus.

FIG. 7 is a cross-sectional view, taken parallel to the longitudinal direction, showing a stopper of the sample preparation apparatus.

FIG. 8 is a cross-sectional view showing the reagent container with the stopper inserted therein.

FIG. 9 is a cross-sectional view showing the reagent container with the stopper inserted therein.

FIG. 10A is a schematic view showing the procedure of using the sample preparation apparatus to collect blood as a liquid sample and performing a process before measuring HbA1c in the blood.

FIG. 10B is a schematic view showing the procedure of using the sample preparation apparatus to collect blood as a liquid sample and performing a process before measuring HbA1c in the blood.

FIG. 10C is a schematic view showing the procedure of using the sample preparation apparatus to collect blood as a liquid sample and performing a process before measuring HbA1c in the blood.

FIG. 10D is a schematic view showing the procedure of using the sample preparation apparatus to collect blood as a liquid sample and performing a process before measuring HbA1c in the blood.

FIG. 11A is a cross-sectional view showing the sample preparation apparatus being used.

FIG. 11B is a cross-sectional view showing the sample preparation apparatus being used.

FIG. 11C is a cross-sectional view showing the sample preparation apparatus being used.

FIG. 12 is a perspective view showing another embodiment of a sample collection device.

FIG. 13 is a side view showing the sample collection device shown in FIG. 12.

FIG. 14 is a side view showing a tip portion of the sample collection device shown in FIG. 12 on an enlarged scale.

FIG. 15 is a front view showing the sample collection device shown in FIG. 12.

FIG. 16 is a perspective view showing another embodiment of a sample collection device.

FIG. 17 is a side view showing the sample collection device shown in FIG. 16.

FIG. 18 is a side view showing the tip portion of the sample collection device shown in FIG. 16.

FIG. 19 is a front view showing the sample collection device shown in FIG. 16.

### DESCRIPTION OF EMBODIMENTS

A capillary may be used as a method for collecting a minute and constant amount of a sample. By using a capillary, it is possible to collect a liquid without using a driving mechanism such as a pump. However, since a sample that is collected by a capillary is typically retained by a capillary force, there is a need, in order to transfer the collected liquid, to exert a force larger than the capillary force upon the liquid, and it may not be easy to discharge the collected liquid sample. In view of such a problem, the present inventor has arrived at a sample collection device having a novel structure. The sample collection device and the sample preparation apparatus of the present disclosure are summarized below.

[Item 1] A sample collection device including:

a grip portion; and

a collecting portion connected to the grip portion,

wherein the collecting portion includes a first surface, and a second surface and a third surface that are adjacent to the first surface with the first surface sandwiched therebetween, and includes a capillary that has openings on the first surface, the second surface and the third surface.

[Item 2] A sample collection device including:

a grip portion; and

a collecting portion connected to the grip portion,

wherein the collecting portion includes a capillary that has openings located at opposite ends thereof and an opening located on a side surface between the opposite ends.

[Item 3] The sample collection device according to item 2, wherein:

the collecting portion includes a first surface, and a second surface and a third surface that are adjacent to the first surface with the first surface sandwiched therebetween; and

the openings of the capillary at the opposite ends are located on the second surface and the third surface, and the opening of the capillary on the side surface is located on the first surface.

[Item 4] A sample collection device including:

a grip portion; and

a collecting portion connected to the grip portion,

wherein the collecting portion includes a first surface and a capillary having a groove shape provided on the first surface and formed with openings at opposite ends thereof.

[Item 5] The sample collection device according to item 4, wherein:

the collecting portion includes a second surface and a third surface that are adjacent to the first surface with the first surface sandwiched therebetween; and

the groove shape includes openings on the second surface and the third surface, and includes an opening of the groove shape on the first surface.

[Item 6] The sample collection device according to any one of items 1 to 5, wherein the capillary has a U-letter shape on a cross section perpendicular to a longitudinal direction thereof.
[Item 7] The sample collection device according to any one of items 1, 3 and 5, wherein the openings located on the second surface and the third surface communicate with each other via the opening located on the first surface.
[Item 8] The sample collection device according to any one of items 1, 3 and 5, wherein the capillary is closed in at least a portion of the first surface.
[Item 9] The sample collection device according to any one of items 1, 3 and 5, wherein a width W of the openings on the second surface and the third surface parallel to the first surface and a depth D thereof perpendicular to the first surface satisfy 0.9≤W/D≤1.1.
[Item 10] The sample collection device according to any one of items 1 to 9, wherein the grip portion extends in a longitudinal direction thereof, and has a polygonal shape on a cross section perpendicular to the longitudinal direction.
[Item 11] The sample collection device according to item 10, wherein the polygonal shape is a pentagonal shape.
[Item 12] The sample collection device according to any one of items 1 to 11, wherein an inner wall of the capillary has a depressed portion or a protruding portion parallel to a longitudinal direction.
[Item 13] The sample collection device according to any one of items 1, 3 and 5, wherein:

the grip portion extends in a longitudinal direction thereof; and

a longitudinal direction of the capillary is perpendicular to the longitudinal direction of the grip portion.

[Item 14] The sample collection device according to item 13, wherein the first surface is perpendicular to the longitudinal direction of the grip portion.
[Item 15] The sample collection device according to item 14, wherein the collecting portion further includes:

a depressed portion located on the first surface; and

a reagent arranged in the depressed portion.

[Item 16] The sample collection device according to item 13, wherein the first surface is non-perpendicular to the longitudinal direction of the grip portion.
[Item 17] The sample collection device according to item 16, wherein the collecting portion further includes:

a fourth surface that is perpendicular to the longitudinal direction of the grip portion and is adjacent to the first surface;

a depressed portion located on the fourth surface; and

a reagent arranged in the depressed portion.

[Item 18] A sample preparation apparatus including:

the sample collection device according to any one of items 1 to 17;

a reagent container having an opening and a bottom portion, and including a chamber that is capable of housing the collecting portion by inserting a part of the sample collection device through the opening, and a through hole that connects the bottom portion with an outside; and

a stopper that can be inserted into the through hole and includes a first end surface and a second end surface located in a longitudinal direction thereof and a side surface located between the first end surface and the second end surface, wherein the stopper includes a first discharge hole that has an opening in the second end surface and extends in the longitudinal direction, and a second discharge hole that has an opening in the side surface and is connected to the first discharge hole,

wherein the stopper is movable between a retain position in which the first surface is located at a bottom of the chamber and a discharge position in which the opening in the side surface is exposed inside the chamber.

Embodiments of the sample collection device and the sample preparation apparatus of the present disclosure will now be described with reference to the drawings. In the following description, embodiments of the present disclosure will be described using, as an example, a sample collection device and a sample preparation apparatus for collecting blood as a liquid sample and measuring the amount of hemoglobin A1c (hereinafter referred to as HbA1c) bound to sugar in blood. However, the sample collection device and sample preparation apparatus of the present disclosure are not limited to the collection of blood, but can be used for the collection of other liquid samples.

To measure HbA1c in blood, for example, a needle is inserted into the fingertip of a finger, causing a minute amount of bleeding, and blood is collected by the sample collection device. Since HbA1c is included in the hemoglobin in the red blood cells of the blood, HbA1c-derived fructosylvalylhistidine is generated by lysing the cell membranes of the red blood cells from the collected blood and breaking down the hemoglobin. The amount of HbA1c in the blood is estimated by quantifying the fructosylbarylhistidine.

The pre-measurement pretreatment before generating the fructosylvalylhistidine described above is preferably performed quickly after collecting the blood. Therefore, the sample collection device for measuring HbA1c is typically used together with the sample preparation apparatus. In the following embodiment, the sample collection device will be first described, and the sample preparation apparatus will be described.

(Sample Collection Device)

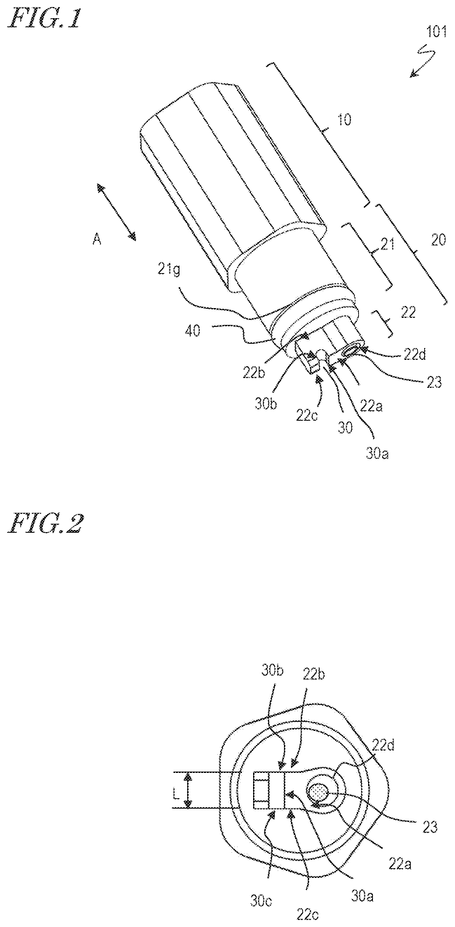

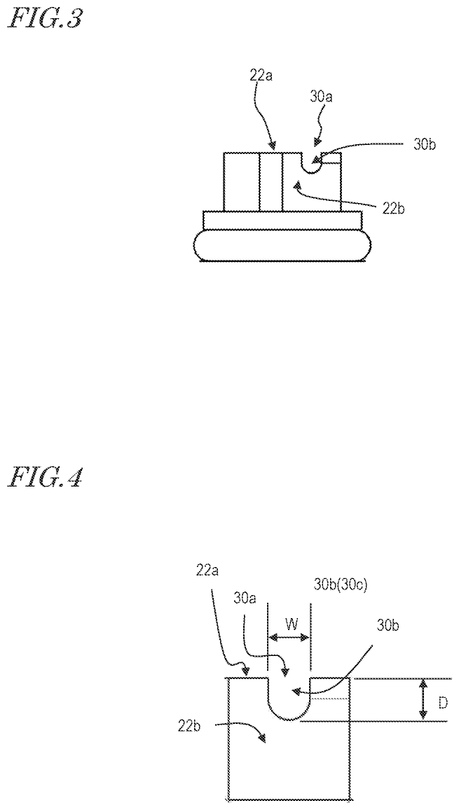

FIG. 1 is a perspective view showing a sample collection device 101 of the present embodiment, and FIG. 2 is a front view thereof as seen from the tip.

The sample collection device 101 includes a grip portion 10 and a collection portion 20. The sample collection device 101 is a device for collecting a minute amount of a liquid, and the amount of the liquid collected is, for example, some tens of microliters or less, and typically 1 to 10 μl or less. Therefore, the sample collection device 101 is sized so that it can be grasped by fingers.

The grip portion 10 is an area for grasping the sample collection device 101, e.g., it is shaped and sized so that it can be easily picked up (easily pinched) with fingertips. Specifically, the grip portion 10 has, for example, a column shape having a length of about 1 to 5 cm in the longitudinal direction A and a width of about 1 to 2 cm across a cross section perpendicular to the longitudinal direction. The cross section perpendicular to the longitudinal direction may have a circular shape, an elliptical shape, etc., or may have a polygonal shape. When it has a polygonal shape, the sample collection device 101 can be easily grasped stably when pinched by fingers because the surfaces to be in contact with fingertips are flat, for example. For example, when the grip portion 10 has a pentagonal shape in the cross section perpendicular to the longitudinal direction, it is possible to ensure sufficiently large surfaces and have many sides with which fingertips are to be in contact. Therefore, for example, when the grip portion 10 is clamped with the thumb, the index finger and the middle finger or when the grip portion is clamped with the thumb and the index finger and a side portion of the middle finger is rested on the grip portion 10, it is possible to select a plurality of sides with which these fingers are to be in contact. Thus, the sample collection device 101 can be stably supported, irrespective of the direction from which the grip portion 10 is pinched. When the cross section of the grip portion 10 has a polygonal shape, the vicinity of the apex of each side may be rounded and the boundary of each side may not be distinct.

When the cross section of the grip portion 10 has a polygonal shape, the grip portion 10 includes a plurality of sides corresponding to the polygonal shape. For example, a particular surface, of these surfaces, may be colored or may be provided with a design pattern to be a marker by using colors or protrusions/depressions. Thus, it is possible for a user to recognize a particular surface, and it is easy for the user to recognize the orientation or the position of a capillary 30 provided in the tip portion to be described below when the grip portion 10 is supported.

The collection portion 20 is connected to one end of the grip portion 10 in the longitudinal direction A, and includes a base portion 21 and a tip portion 22. The capillary (capillary space) 30 for collecting a liquid is provided in the tip portion 22. The base portion 21 separates the tip portion 22 from the grip portion 10 in order to prevent the tip portion 22 from being visually obstructed by fingers when the grip portion 10 is supported by fingers. The base portion 21, for example, has a cylindrical shape. In the present embodiment, a groove 21g is provided along the circumferential direction of the side of the base portion 21, and an O-ring 40 is arranged in the groove 21g. The base portion 21 does not need to be provided depending on the shape of the grip portion 10. For example, where one side of the grip portion 10 that is connected to the collection portion 20 is configured to extend narrow and not to support fingers, the tip portion 22 may be directly connected to the grip portion 10.

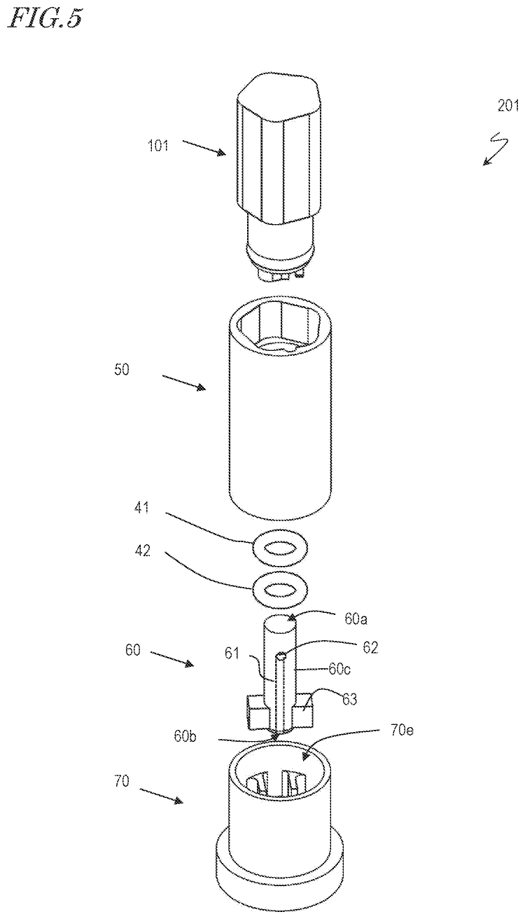

FIG. 3 and FIG. 4 are side views showing the tip portion on an enlarged scale. The tip portion 22 includes the capillary 30 as described above. The capillary 30 is arranged in the tip portion 22 so as to have openings 30b and 30c that are located on the opposite sides, and an opening 30a that is located on a side surface. Specifically, the capillary 30 has a groove shape and the tip portion 22 has a first surface 22a, and the capillary 30 is provided on the first surface 22a so that the opening 30a, which is an opening of the groove, is located on the first surface 22a. In the present embodiment, the first surface 22a is perpendicular to the longitudinal direction of the grip portion 10. Herein, “perpendicular” refers to having an angle of 80° to 100°.

More specifically, the tip portion 22 has a second surface 22b and a third surface 22c that are adjacent to the first surface 22a with the first surface 22a sandwiched therebetween, and the opening 30b and the opening 30c of the capillary 30 are provided on the second surface 22b and the third surface 22c.

In the present embodiment, the opening 30b is connected to the opening 30c by the opening 30a, and the opening 30b and the opening 30c communicate with each other. In other words, the capillary 30 has the opening 30c in all of the portion in contact with the first surface 22a. However, the capillary 30 may be closed in at least a portion of the first surface 22a. Thus, it is possible to vary the size of the opening 30a so as to adjust the capillary force in the capillary 30 and to adjust the retention force to hold the liquid sample in the capillary 30.

The capillary 30 has its longitudinal direction perpendicular to the longitudinal direction A of the grip portion 10, and extends in the longitudinal direction. The capillary 30 preferably has a U-letter shape on a cross section perpendicular to the longitudinal direction. By having a U-letter shape, with a straight line portion and a curve portion of the cross section, it is possible to adjust the direction and the magnitude of the capillary force and the discharge of the liquid sample retained therein. With the bottom of the U-letter shape, i.e., the inner surface on the side farther away from the opening 30a of the first surface 22a, being a curved surface, it is less likely that bubbles are produced when a liquid sample is drawn by the capillary force. Therefore, it is possible to possible to more accurately measure a predetermined amount of a liquid sample.

The opening 30b and the opening 30c preferably have the same shape as the cross section. The width W parallel to the first surface 22a of the openings 30b and 30c and the depth D perpendicular to the first surface 22a are preferably approximately equal to each other. Specifically, the width W and the depth D preferably satisfy the relationship of 0.9≤W/D≤1.1.

The capillary 30 preferably has a size such that there is exerted a capillary force appropriate for the liquid to be collected. For example, when the liquid sample is blood, the width W and the depth D are preferably in the range of 800 μm to 1 mm, for example. The length L of the capillary 30 in the longitudinal direction is preferably in the range of 2 mm to 4 mm. The adjustment of the amount of a liquid sample to be collected can be adjusted primarily by the length L in the longitudinal direction.

The inner wall of the capillary 30 may have at least one of a linear depressed portion and a linear protruding portion extending parallel to the longitudinal direction. The provision of a depressed portion or a protruding portion makes it easier for a liquid sample to flow in through the opening 30b and the opening 30c and increases the surface area of the inner wall by virtue of the protrusion/depression, thereby increasing the retention force of the capillary force, thus making it possible to adjust the liquid sample retention characteristic or the liquid sample discharge characteristic. Such a linear protrusion/depression may be a resin molding flow line that occurs when a resin flows in the mold when producing the sample collection device 101 by way of extrusion molding using a mold, for example.

If there is a need to perform a pre-treatment on a liquid sample in order to measure a particular component in the liquid sample after the liquid sample is collected by the sample collection device 101, a reagent for use in the pre-treatment may be arranged in the tip portion 22. Specifically, a depressed portion 22d may be provided on the first surface 22a, and a reagent 23 may be arranged in the depressed portion 22d. More specifically, the sample collection device 101 may further include a protease, which is a hemoglobin breakdown enzyme, as the reagent 23. The depressed portion 22d may be arranged in proximity to the opening 30a of the capillary 30.

With the sample collection device 101, the capillary 30 has openings in three directions. Thus, when collecting a liquid sample, the liquid sample can be sucked in through the three openings, and the air that has filled the capillary 30 can be discharged through one of the openings in the three directions when the liquid sample flows into the capillary 30. For example, if blood is drawn through the opening 30a located on the first surface 22a, the air that has filled the capillary 30 is discharged through the opening 30b and the opening 30c, which are on the opposite ends. Therefore, the entire capillary 30 is filled with blood quickly and without producing bubbles therein. Thus, it is easy to accurately measure a constant amount of a liquid sample.

When blood that is filling the capillary 30 is discharged (released) from the sample collection device 101, since there are openings in three directions, the liquid sample retained therein can be easily discharged through the openings. Particularly, since the opening 30b and the opening 30c, which are located at the opposite ends of the capillary 30, are provided on the second surface 22b and the third surface 22c, when the sample collection device 101 is shaken in the longitudinal direction A while holding the grip portion 10, for example, an inertial force acts upon the liquid sample supported in the capillary 30, and the liquid sample is easily discharged through the opening 30b and the opening 30c. Thus, with the sample collection device 101 of the present embodiment, it is possible to easily release the collected liquid sample.

The capillary 33 extends perpendicular to the longitudinal direction A of the grip portion 10. When the collected sample liquid is discharged into a liquid such as a pre-treatment solution, the entirety of the capillary 30 can be immersed in the pre-treatment solution even if the amount of the pre-treatment solution, or the like, is small and therefore the liquid level of the pre-treatment solution is low. Thus, the collected liquid sample can be discharged into a small amount of a solution such as a pre-treatment solution. Where there is a need for a pre-treatment, it is possible to increase the concentration of the sample in the solution to be prepared.

Moreover, a reagent for use in a pre-treatment can be arranged in proximity to the capillary 33. Therefore, as the reagent dissolves into a pre-treatment solution, the sample discharged into the pre-treatment solution and the reagent can be brought in proximity to each other, thereby realizing quick reaction.

(Sample Preparation Apparatus)

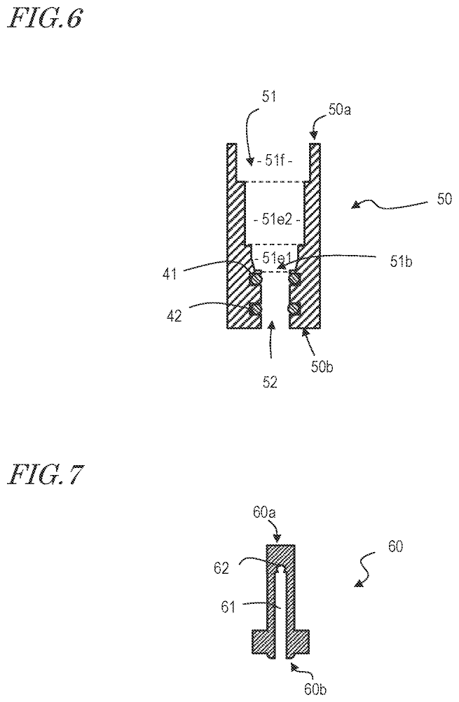

An embodiment of the sample preparation apparatus will be described. FIG. 5 is an exploded perspective view of a sample preparation apparatus 201. The sample preparation apparatus 201 includes the sample collection device 101, the reagent container 50, a stopper 60 and a cover 70. The sample collection device 101 has a structure described above. FIG. 6 and FIG. 7 are cross-sectional views, taken parallel to the longitudinal direction, showing the reagent container 50 and the stopper 60.

The reagent container 50 has a column shape having an upper surface 50a and a bottom surface 50b, and has a chamber 51 and a through hole 52 that house therein at least the collection portion 20 of the sample collection device 101. The chamber 51 includes an opening 51o in the upper surface 50a, and the sample collection device 101 is inserted into the chamber 51 through an opening 51c. The chamber 51 also includes a bottom portion 51b. In the present embodiment, the chamber 51 includes a first portion 5101 into which the tip portion 22 of the collection portion 20 of the sample collection device 101 is inserted, a second portion 51e2 into which the base portion 21 is inserted, and a third portion 51f into which a part of the grip portion 10 is inserted. The through hole 52 is provided in the bottom surface 50b, and connects the bottom portion 51b of the chamber 51 with the outside. Grooves are provided along the circumferential direction on the side surface of the through hole 52, and O-rings 41 and 42 are arranged in the grooves.

The stopper 60 has a column shape having a first end surface 60a, a second end surface 60b, and a side surface 60c that is located between the first end surface 60a and the second end surface 60b, and the stopper 60 is capable of being inserted into the through hole 52 of the reagent container 50.

The second end surface 60b is provided with a first discharge hole 61 that extends along the longitudinal direction of the column shape. It is also provided with a second discharge hole 62 that has an opening in the side surface 60c, extends perpendicular to the longitudinal direction, and is connected to the first discharge hole 61. A protruding portion 63 is provided on the side surface 60c in the vicinity of the second end surface 60b.

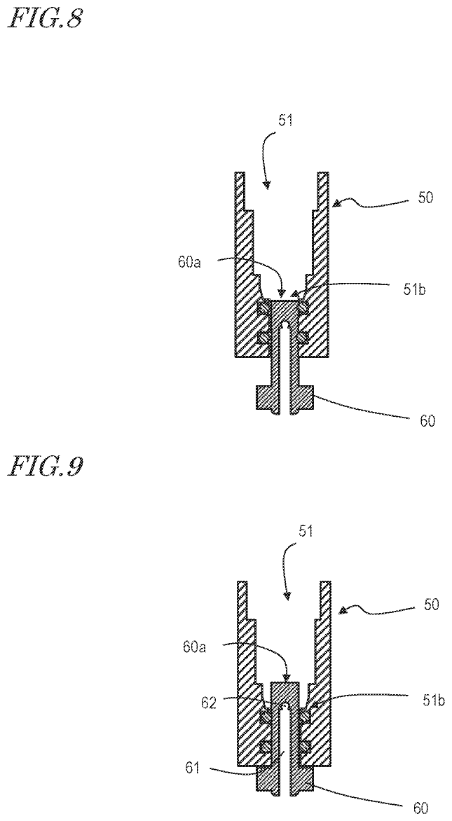

The stopper 60 is used while it is inserted into the reagent container 50. FIG. 8 and FIG. 9 are cross-sectional views showing the stopper 60 inserted into the through hole 52 of the reagent container 50. The stopper 60 is movable between the retain position in which the first end surface 60a is located at the bottom portion 51b of the chamber 51 as shown in FIG. 8, and the discharge position in which the stopper 60 is inserted further into the through hole 52 so that the first end surface 60a rises past the bottom portion 51b and so that the opening of the second discharge hole 62 provided in the side surface 60c is located in the chamber 51. In order to prevent the stopper 60 from being moved inadvertently, the stopper 60 may be configured so that the stopper 60 can move in the longitudinal direction only when the stopper 60 is turned around the longitudinal axis to be aligned at a particular angular position.

As will be described in detail below, when the stopper 60 is in the retain position, a reagent solution for lysing the sample is retained in the chamber 51. When the stopper 60 is in the discharge position, the reagent solution in the chamber 51 is discharged to the outside through the second discharge hole 62 and the first discharge hole 61. Even though the stopper 60 is movably inserted into the through hole 52, since the O-rings 41 and 42 are arranged in the through hole 52, the gap between the through hole 52 and the stopper 60 is sealed, and the liquid retained in the chamber 51 is prevented from leaking through the through hole 52.

The cover 70 has a space 70e and houses the stopper 60 inserted into the reagent container 50 to support the reagent container 50.

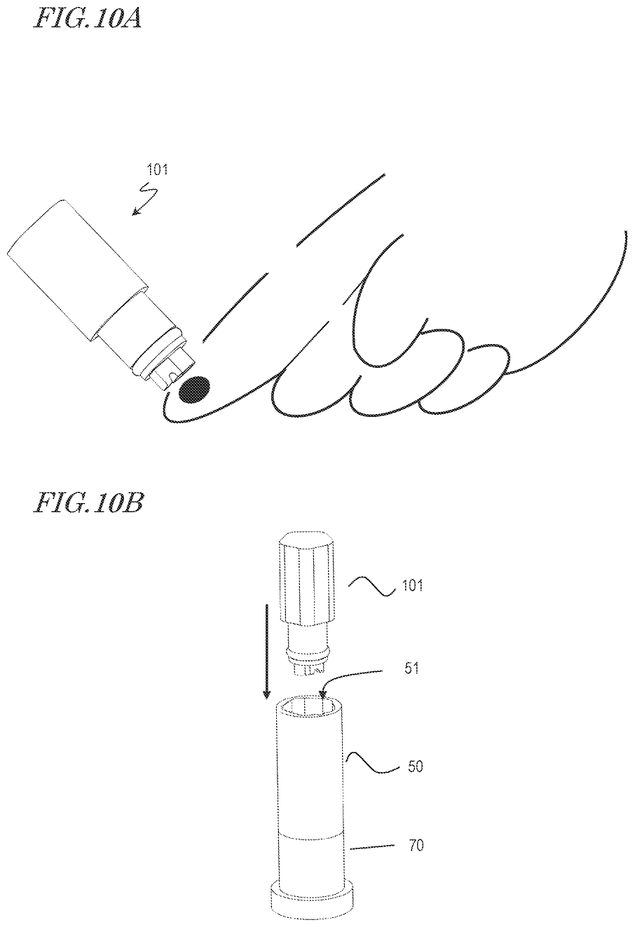

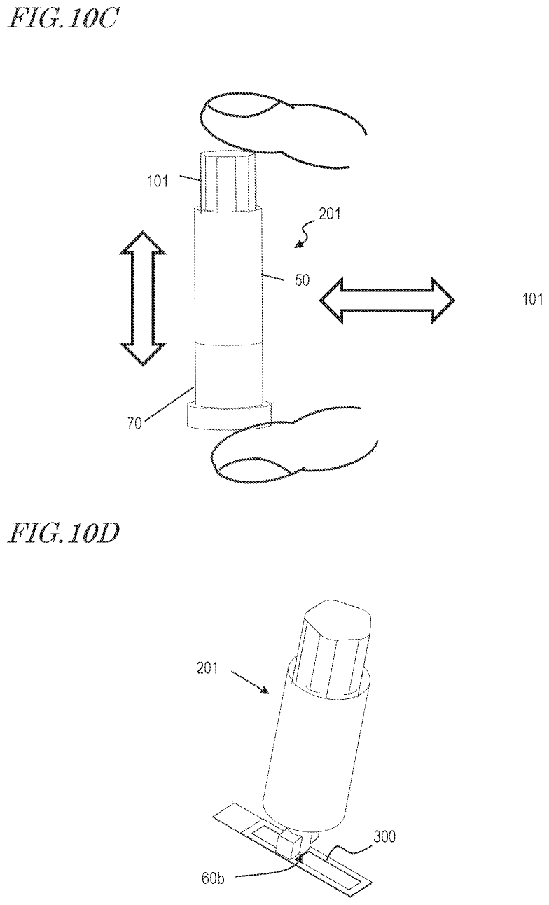

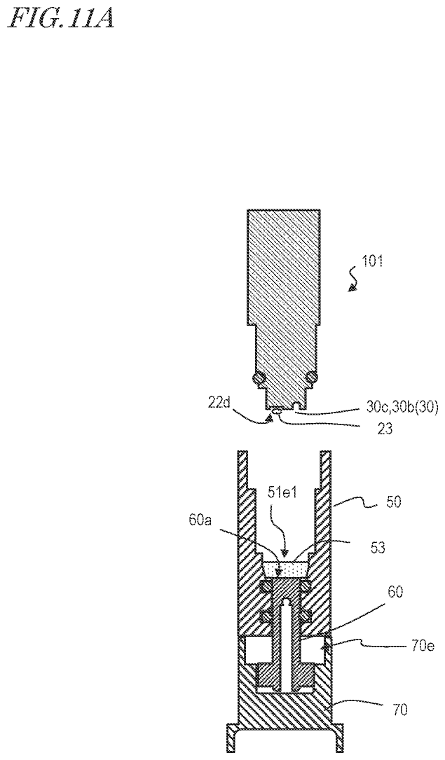

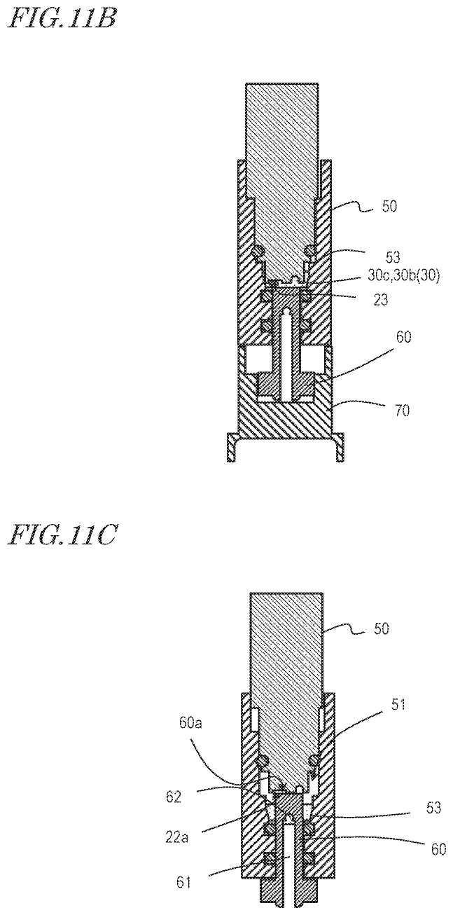

Next, the use of the sample preparation apparatus 201 will now be described with reference to FIG. 10A to FIG. 10D and FIG. 11A to FIG. 11C. FIG. 10A to FIG. 10D are schematic views showing the procedure of using the sample preparation apparatus 201 to collect blood as a liquid sample and performing a pre-treatment before measuring HbA1c in the blood, and FIG. 11A to FIG. 11C are cross-sectional views showing the sample preparation apparatus 201 being used.

As shown in FIG. 11A, a protease, which is a hemoglobin breakdown enzyme, is arranged as the reagent 23 in the depressed portion 22d of the sample collection device 101. The stopper 60 is inserted into the through hole 52 of the reagent container 50 so as to be in the retain position. The reagent container 50 is supported by the cover 70 so that the stopper 60 is housed in the space 70e.

The chamber 51 of the reagent container 50 retains a lysate 53 including a surfactant as a hemolytic agent to lyse red blood cells in the blood.

First, as shown in FIG. 10A, a needle is inserted into a fingertip to cause a minute amount of bleeding and bring blood into contact with one of the openings 30a, 30b and 30c in the tip portion 22 of the sample collection device 101, thereby sucking blood into the capillary 30. Next, the collection portion 20 of the sample collection device 101 is inserted into the opening of the chamber 51 of the reagent container 50, as shown in FIG. 10B, and the cover 70 and the sample collection device 101 are pinched by fingers and shaken in the up-down direction (the longitudinal direction) and in the left-right direction, as shown in FIG. 10C.

As shown in FIG. 11B, the collection portion 20 of the sample collection device 101 is inserted into the chamber 51, thereby immersing a part or whole of the tip portion 22 in the lysate 53. At this point, if the openings 30b and 30c of the capillary 30 are entirely immersed in the lysate 53, the blood retained in the capillary 30 is caused by the shaking to effectively dissolve into the lysate 53. As is clear from FIG. 11B, since the capillary 30 extends in the horizontal direction, the entirety of the capillary 30 can be immersed in the lysate 53 even if the amount of the lysate 53 is small and the liquid level is low. Since the openings 30a, 30b and 30c (FIG. 2) are provided in three directions of the capillary 30, the retained blood is in contact with the lysate 53 over a large area, and the retained blood is discharged from the capillary 30 to the lysate 53 therearound by virtue of the inertial force acting upon the blood through the shaking and the shaking of the lysate 53 produced by the shaking. At this point, the reagent 23 retained in the depressed portion 22d also dissolves into the lysate 53. When the blood dissolves into the lysate 53, the surfactant in the lysate 53 and the red blood cells react with each other, and hemoglobin dissolves from red blood cells into the lysate 53. Hemoglobin further reacts with the reagent 23 in the lysate 53, thereby producing fructosylvalylhistidine.

Thereafter, as shown in FIG. 10C, the cover 70 is removed, and the second end surface 60b of the stopper 60 is pressed against a sample introducing portion 300 of the measurement apparatus. As the second end surface 60b of the stopper 60 is pressed against the sample introducing portion 300, the stopper 60 is inserted into the through hole 52 of the reagent container 50, and the first end surface 60a contacts the first surface 22a of the tip portion 22 of the sample collection device 101, thereby pushing up the sample collection device 101, as shown in FIG. 11C. When the protruding portion 63 of the stopper 60 contacts the bottom surface 50b of the reagent container 50, the opening of the second discharge hole 62 of the stopper 60 is exposed inside the chamber 51. Thus, the lysate 53 passes through the second discharge hole 62 and the first discharge hole 61 to be introduced to the sample introducing portion 300 of the measurement apparatus.

The measurement apparatus guides the lysate 53, which has been introduced from the sample introducing portion 300, to a sensor and measures the HbA1c concentration and the total hemoglobin concentration through an electrochemical reaction.

### Other Embodiments of Sample Collection Device

Various modifications can be made to the sample collection device.

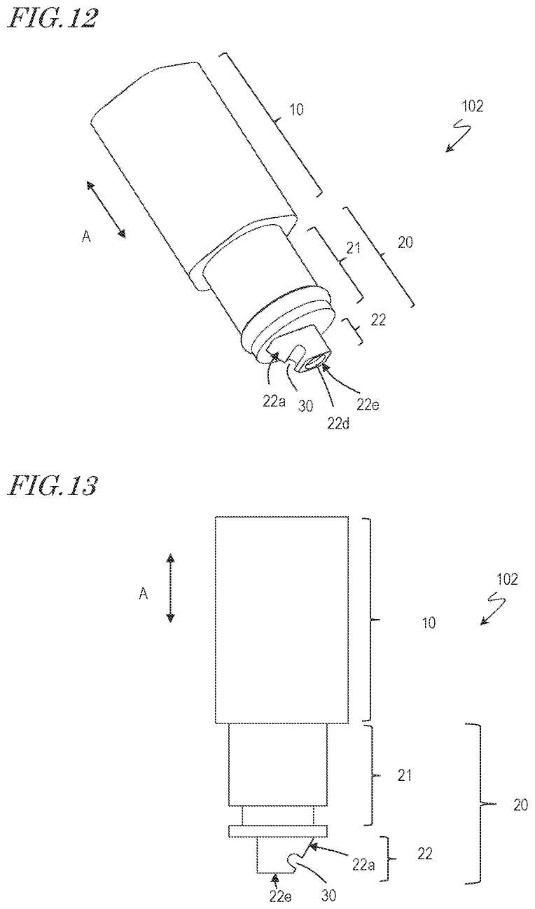

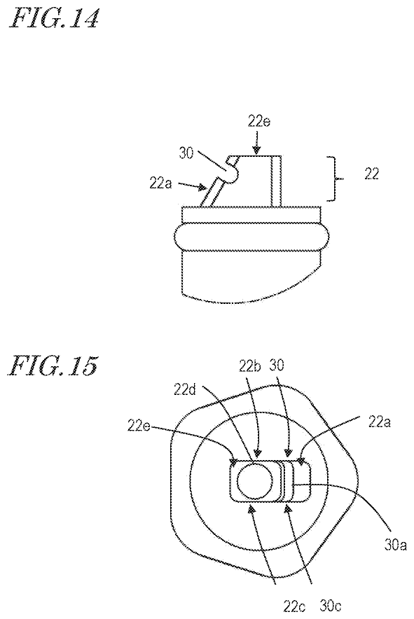

FIG. 12 is a perspective view showing a sample collection device 102 according to another embodiment, and FIG. 13 is a side view showing the sample collection device 102. FIG. 14 is a side view showing a tip portion of the sample collection device 102 on an enlarged scale, and FIG. 15 is a front view showing the sample collection device 102.

The sample collection device 102 is different from the sample collection device 101 in that the first surface on which the capillary 30 is provided is non-perpendicular to the longitudinal direction A of the grip portion 10. At the tip portion 22 of the sample collection device 102, the collection portion 20 has a fourth surface 22e adjacent to the first surface 22a, and the fourth surface 22e is provided with the depressed portion 22d for retaining a reagent. The fourth surface 22e is perpendicular to the longitudinal direction A of the grip portion 10, for example. The capillary 30 is preferably on a portion of the first surface 22a that is in proximity to the boundary between the first surface 22a and the fourth surface 22e.

With the first surface 22a being non-perpendicular to the longitudinal direction A, the tip portion 22 has a shape that is tapered toward the tip as seen from the side. Therefore, when collecting blood from a fingertip, for example, it is easy for the user to recognize the portion where the capillary 30 is located, and it is easy for the user to accurately bring the portion where the capillary 30 is located into contact with blood on the fingertip. Thus, it is possible to enhance the operability and utility for the operator when collecting blood.

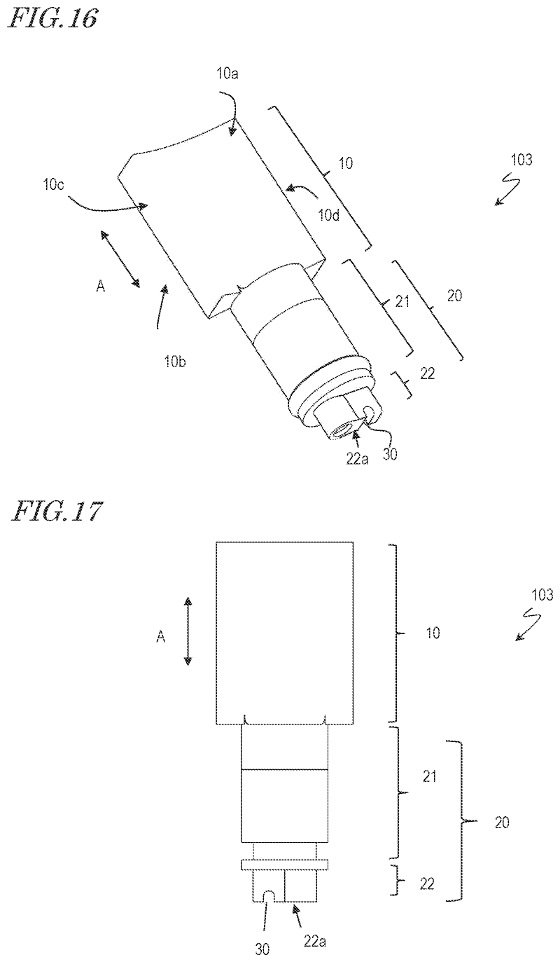

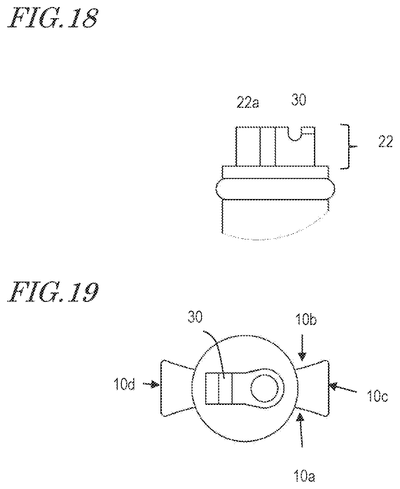

FIG. 16 is a perspective view showing a sample collection device 103 according to another embodiment, and FIG. 17 is a side view showing the sample collection device 103. FIG. 18 is a side view showing a tip portion of the sample collection device 103 on an enlarged scale, and FIG. 19 is a front view showing the sample collection device 103.

The sample collection device 103 differs from the sample collection device 101 in that the sample collection device 103 includes a grip portion 10 of a different shape. Specifically, the grip portion 10 of the sample collection device 103 has a generally rectangular shape on a cross section perpendicular to the longitudinal direction. The grip portion 10 includes a pair of first side surfaces 10a and 10b and a pair of second side surfaces 10c and 10d, and the area of the first side surfaces 10a and 10b is greater than the area of the second side surfaces 10c and 10d. The first side surface 10a and the first side surface 10b are located on the opposite side from each other, and the second side surface 10c and the second side surface 10c are similarly located on the opposite side from each other. The first side surfaces 10a and 10b are generally perpendicular to the longitudinal direction of the capillary 30.

When the sample collection device 103 is used, since the first side surfaces 10a and 10b are large, the user places the thumb and the index finger, for example, on the first side surfaces 10a and 10b so as to pinch the first side surfaces 10a and 10b with no particular instruction. When the user shakes the sample collection device 103 in this state, the sample collection device 103 is shaken generally along the longitudinal direction of the capillary 30. When shaken in this direction, the collected liquid sample is likely to be discharged through the openings 30b and 30c by virtue of the inertial force. Therefore, with the sample collection device 103, the user can shake the sample collection device 103 in such a direction that the collected liquid sample is likely to be discharged. Therefore, a pre-treatment for a sample can be completed by a shorter shaking operation, resulting in a good operability.

### INDUSTRIAL APPLICABILITY

The sample collection device and the sample preparation apparatus of the present disclosure are capable of collecting various liquid samples such as blood and adjusting the liquid samples after collection, and are thus suitable for component analysis of blood, etc.

### REFERENCE SIGNS LIST

10 Grip portion
10a, 10b First side surface
10c, 10d Second side surface
20 Collecting portion
21 Base portion
21g Groove
22 Tip portion
22a First surface
22b Second surface
22c Third surface
22d Depressed portion
22e Fourth surface
23 Reagent
30 Capillary
30a to 30c Opening
33 Capillary
40, 41 O-ring
50 Reagent container
50a Upper surface
50b Bottom surface
51 Chamber
51c Opening
51e1 First portion
51e2 Second portion
51f Third portion
52 Through hole
53 Lysate
60 Stopper
60a First end surface
60b Second end surface
60c Side surface
61 First discharge hole
62 Second discharge hole
63 Protruding portion
70 Cover
70e Space
101 to 103 Sample collection device
201 Sample preparation apparatus
300 Sample introducing portion

1. A sample collection device comprising:
a grip portion; and
a collecting portion connected to the grip portion,
wherein the collecting portion includes a first surface, a second surface and a third surface that are adjacent to the first surface with the first surface sandwiched therebetween, and a capillary that has openings on the first surface, the second surface and the third surface.

2. A sample collection device comprising:
a grip portion; and
a collecting portion connected to the grip portion,
wherein the collecting portion includes a capillary that has openings located at opposite ends thereof and an opening located on a side surface between the opposite ends.

3. The sample collection device according to claim 2, wherein:
the collecting portion includes a first surface, and a second surface and a third surface that are adjacent to the first surface with the first surface sandwiched therebetween; and
the openings of the capillary at the opposite ends are located on the second surface and the third surface, and the opening of the capillary on the side surface is located on the first surface.

4. A sample collection device comprising:
a grip portion; and
a collecting portion connected to the grip portion,
wherein the collecting portion includes a first surface and a capillary having a groove shape provided on the first surface and formed with openings at opposite ends thereof.

5. The sample collection device according to claim 4, wherein:
the collecting portion includes a second surface and a third surface that are adjacent to the first surface with the first surface sandwiched therebetween; and
the groove shape includes openings on the second surface and the third surface, and includes an opening of the groove shape on the first surface.

6. The sample collection device according to claim 1, wherein the capillary has a U-letter shape on a cross section perpendicular to a longitudinal direction thereof.

7. The sample collection device according to claim 1, wherein the openings located on the second surface and the third surface communicate with each other via the opening located on the first surface.

8. The sample collection device according to claim 1, wherein the capillary is closed in at least a portion of the first surface.

9. The sample collection device according to claim 1, wherein a width W of the openings on the second surface and the third surface parallel to the first surface and a depth D thereof perpendicular to the first surface satisfy 0.9≤W/D≤1.1.

10. The sample collection device according to claim 1, wherein the grip portion extends in a longitudinal direction thereof, and has a polygonal shape on a cross section perpendicular to the longitudinal direction.

11. The sample collection device according to claim 10, wherein the polygonal shape is a pentagonal shape.

12. The sample collection device according to claim 1, wherein an inner wall of the capillary has a depressed portion or a protruding portion parallel to a longitudinal direction.

13. The sample collection device according to claim 1, wherein:
the grip portion extends in a longitudinal direction thereof; and
a longitudinal direction of the capillary is perpendicular to the longitudinal direction of the grip portion.

14. The sample collection device according to claim 13, wherein the first surface is perpendicular to the longitudinal direction of the grip portion.

15. The sample collection device according to claim 14, wherein the collecting portion further includes:
a depressed portion located on the first surface; and
a reagent arranged in the depressed portion.

16. The sample collection device according to claim 13, wherein the first surface is non-perpendicular to the longitudinal direction of the grip portion.

17. The sample collection device according to claim 16, wherein the collecting portion further includes:
a fourth surface that is perpendicular to the longitudinal direction of the grip portion and is adjacent to the first surface;
a depressed portion located on the fourth surface; and
a reagent arranged in the depressed portion.

18. A sample preparation apparatus comprising:
the sample collection device according to claim 1;
a reagent container having an opening and a bottom portion, and including a chamber that is capable of housing the collecting portion by inserting a part of the sample collection device through the opening, and a through hole that connects the bottom portion with an outside; and
a stopper that can be inserted into the through hole and includes a first end surface and a second end surface located in a longitudinal direction thereof and a side surface located between the first end surface and the second end surface, wherein the stopper includes a first discharge hole that has an opening in the second end surface and extends in the longitudinal direction, and a second discharge hole that has an opening in the side surface and is connected to the first discharge hole,
wherein the stopper is movable between a retain position in which the first surface is located at a bottom of the chamber and a discharge position in which the opening in the side surface is exposed inside the chamber.
